(19) United States
(12) Patent Application Publication
(10) Pub. No.: US 2013/0034211 A1
(43) Pub. Date: Feb. 7, 2013

(54) Radiotherapy

(71) Applicant: ELEKTA AB (PUBL), Stockholm (SE)

(72) Inventor: Bengt Åke Claesson, Uppsala (SE)

(73) Assignee: ELEKTA AB (PUBL), Stockholm (SE)

(21) Appl. No.: 13/647,512

(22) Filed: Oct. 9, 2012

Related U.S. Application Data
(63) Continuation of application No. PCT/EP2011/002130, filed on Apr. 28, 2011.

(51) Int. Cl. A61N 5/10

(52) U.S. Cl. 378/65

(57) ABSTRACT

Radiotherapy apparatus is disclosed, comprising a radiation source capable of emitting a beam of therapeutic radiation along a beam axis, collimation apparatus for delimiting the beam and comprising (i) a block of sufficient width to extend across the width of the beam, selectively movable into the beam from a first side of the beam axis, and (ii) an array of individually moveable elongate narrow leaves arranged side-by-side in a direction perpendicular to the beam, each being moveable longitudinally into the beam from a second and opposing side of the beam axis, in which there is no array of individually moveable elongate narrow leaves arranged side-by-side in a direction perpendicular to the beam moveable longitudinally into the beam from the first side of the beam axis. Thus, there is in effect a single bank of MLC leaves on one side of the aperture and a block collimator on the other.

This application is a continuation of Patent Cooperation Treaty Patent Application PCT/EP2011/002130, filed Apr. 28, 2011, which is incorporated herein by reference.

### FIELD OF THE INVENTION

The present invention relates to improvements in the apparatus and related processes for the delivery of radiotherapy.

### BACKGROUND ART

Radiotherapy consists of the treatment of tumours and other lesions by directing harmful radiation towards the site of the lesion. That radiation is partially absorbed by the lesion, causing cell damage which inhibits growth of the lesion and/or causes it to reduce in size.

The radiation is also capable of causing harm to surrounding healthy tissue, albeit at a slightly lesser rate in the case of cancerous lesions. Whilst it is impossible to completely prevent the irradiation of healthy tissue, given that some tissue will be in front of or behind the lesion, the amount of healthy tissue that is irradiated is generally minimised by careful collimation of the beam and planning of the treatment.

FIG. 1 shows the general layout of the essential functional parts of a radiotherapy source. High-energy electrons are directed toward an x-ray target 1 in order to generate therapeutic x-rays of (typically) 1-4 MeV. These are delimited by a primary collimator 1 comprising a substantial block of x-ray attenuating material such as tungsten with a conical through-aperture centred on the x-ray source 1. Filters 3 and 4 then remove unwanted wavelengths from the beam and flatten its intensity profile. An ion chamber 5 allows the beam properties to be monitored, and a wedge filter 6 provides further control of the beam properties.

The beam thus created has a standard shape, which is then tailored to the specific needs of the treatment being delivered at that instant by collimators 7, 8, 9. These consist of a pair of MLC banks 7, designed to collimate the beam to any desired irregular shape (such as the outline of a tumour). It consists of two opposing banks of leaves, either side of the central axis of the beam. Each of the two banks has a plurality of tungsten leaves (such as 40, 60, or 80 leaves per side, i.e. 80, 120 or 160 leaves in total) arranged side by side. Each leaf consists of an elongate, thin, flat generally rectangular shape, arranged so that the short side of the rectangle is arranged generally parallel to the beam axis and the long side extends generally transverse to the beam axis. Within each bank, the leaves are side-by-side and are all individually moveable in the direction of their long edges. Thus, viewed along the direction of the beam, the leaves each present a long narrow shadow that can be extended into the beam aperture. Each bank provides an array of leaves that can be selectively extended or retracted so as to define a desired edge shape. Between them, the two banks allow substantially any shape to be defined.

FIG. 2 shows a schematic arrangement of a single bank of leaves, consisting of an array of individual leaves 10, 12, 14 etc. These are each about 10-15 cm deep in the direction of the beam B in order to provide adequate attenuation of the beam, and about 30-40 cm long in the longitudinal direction L, perpendicular to the beam direction B, so that they can be extended fully across the beam aperture. Each leaf is also approximately 2 mm thickness, in the direction N that is perpendicular to both the bean direction B and the longitudinal direction L; arrayed side-by-side in the N direction, this allows n leaves to offer a correspondingly magnified resolution at the patient.

In practice, the leaf array shown in FIG. 2 is universally combined with a further bank of leaves opposing the first (as shown in FIG. 1), so as to provide a symmetrical aperture definition.

Multi-leaf collimators have a number of drawbacks. In particular, there is a small but finite amount of leakage of radiation through the very small gaps between the leaves. The need for the leaves to move independently of each other means that there must be a small clearance between them in order to eliminate friction; this clearance allows a leakage path. The amount of leakage can be reduced by various means, including slight misalignment of the leaves to the radiation beam, and interlocking formations on the sides of the leaves. However, there is usually still enough leakage for manufacturers to provide additional collimators in the form of so-called “block collimators”, substantial tungsten blocks similar in depth to the leaves, and extending across the entire width of the radiation aperture. These extend into the beam substantially parallel to the MLC leaves, and can be positioned so that their forward edge is just short of the least-extended leaf. They can be positioned above or below the MLC along the beam axis.

This is shown in FIG. 3, in which the leaf banks are viewed along the beam direction B. The left-hand leaf bank 20 includes leaves 10, 12, 14 etc., and faces a right-hand leaf bank 22 including a like set of leaves. It should be noted that “left” and “right” are used for convenience, as in practice the radiation head containing the MLC can be rotated bodily around the patient and also around the beam axis, so the absolute position of each ban may vary considerably in use. Thus, left and right refer to the positions of the leaf banks in FIG. 3 and does not imply that these positions are maintained at all times. In this instance, the left bank 20 has been adjusted so as to define the left edge of a desired beam shape 24, with the right bank 22 defining the right edge of the beam shape 24. A left block collimator 26, shown in outline as it may be above or below the leaf banks, is advanced into the beam aperture so that its leading edge 28 lies just behind the leading edge of the least-advanced MLC leaf 14 (highlighted). Likewise, a right block collimator 30 is advanced into the beam aperture so that its leading edge lies just behind the leading edge of the least-advanced MLC leaf (also highlighted). In this way, leakage between the MLC leaves is minimised.

FIG. 1 shows the block collimator 8, beneath the MLC banks 7. In practice, the block collimator can alternatively be located above the MLC and suitable geometric adjustments made to allow for the beam divergence. One side of a second block collimator 9 is also shown, operating in the transverse axis to that of the MLC leaves 7 in order to delimit the edges of the treatment field.

A multi-leaf collimator can thus be used to deliver a radiation field with a desired outline. This can, for example, conform to the outline of the tumour (or, in practice, the tumour plus a surrounding volume to allow for movement and for measurement tolerances).

It can also be used to deliver a radiation field with a desired dose pattern, i.e. a field in which parts receive a lower dose and other parts receive a higher dose. This is illustrated in FIGS. 4 and 5. A first approach to doing this is shown in FIG. 4; a desired dose profile is shown in graph 32. Normally, the dose profile would be a three-dimensional profile, with a dose varying across the two spatial dimensions of the beam aperture. Graph 32 shows the section though that profile corresponding to one leaf pair, such as leaf 14 of the left-hand array 20 and the opposing leaf 14a of the right-hand array 22. To build up the dose profile shown in graph 32, leaves 14 and 14a are advanced into the field so that they bound the outer extremities of the area that is to receive a non-zero dose. The beam is then switched on, and a dose begins to build in the area between the leaves 14, 14a. After an initial time period P1 has passed during which a dose D1 has been delivered, the leaves 14, 14a are advanced further so that they cover the outermost areas 34, 36 where no more than D1 is needed, and just bound the area where the next higher dose D2 is needed. Again, a time period P2 is allowed to elapse after which the total dose delivered during P1 and P2 is equal to D2. The leaves are then advanced further to place areas 38, 40 where no more than D2 is needed in shadow, and delimit areas 42, 44 where the next highest dose D3 is needed. A further time period P3 is allowed so that the dose in the areas still exposed can build to D3, and the leaves are advanced again. In this example, there is only one central portion 46 in which a dose D4 is needed, so during the final period P4 this region is allowed to acquire dose D4, after which the beam is switched off.

This method allows a varying dose distribution such as that shown in graph 32 to be built up, with the leaves moving during the treatment so as to build up the required pattern. In concert with the other 39, 79 or 159 leaves moving according to the same method, a two-dimensional pattern can be delivered across the beam aperture.

For dose patterns that are not bell-shaped as in FIG. 4, or where the maximum dose differs between different leaf pairs, the method shown in FIG. 5 offers more flexibility and is the one usually adopted. In this pattern, the leading leaf 14a starts at some point in the middle of the field, with the trailing leaf 14 at the edge of the area to be irradiated. The beam is then activated. The leading leaf 14a is withdrawn and the trailing leaf 14 is advanced across the beam aperture as and when necessary to build up the dose pattern of graph 34. FIG. 5 shows the movements required, and it will be seen that each area is exposed for a time sufficient to deliver the correct dose.

MLC-based intensity modulation of this type is referred to as “IMRT”, “Intensity Modulated Radio Therapy”. It can be delivered as “Step-and-shoot” IMRT, which means that while the leaf pairs are being moved between discrete positions, the beam is turned off, or as “Dynamic” IMRT which means the leaves are moving continuously with the beam on throughout the entire delivery session. It is also advantageous to rotate the gantry during dynamic IMRT, which is referred to as “Volumetric Modulated Arc Therapy” or “VMAT”. For IMRT and IMRT-based techniques such as VMAT, it is usual for the leaves and the dose rates to be controlled by a suitably programmed computer implementing a treatment plan developed by a treatment planning computer.

### SUMMARY OF THE INVENTION

The present invention provides a radiotherapy apparatus comprising a radiation source capable of emitting a beam of therapeutic radiation along a beam axis, collimation apparatus for delimiting the beam and comprising (i) a block of sufficient width to extend across the width of the beam, selectively movable into the beam from a first side of the beam axis, and (ii) an array of individually moveable elongate narrow leaves arranged side-by-side in a direction perpendicular to the beam, each being moveable longitudinally into the beam from a second and opposing side of the beam axis, in which there is no array of individually moveable elongate narrow leaves arranged side-by-side in a direction perpendicular to the beam moveable longitudinally into the beam from the first side of the beam axis. Thus, there is in effect a single bank of MLC leaves on one side of the aperture and a block collimator on the other.

This is still perfectly usable for IMRT-based treatments by simply assuming that the leaves on one bank of the MLC need to be aligned at all times. The times and positions of the leaves on the opposing bank can be adjusted accordingly, meaning that one “bank” of the MLC comprises a set of leaves that present a straight, uniform front at all relevant times. This “bank” can then be replaced with a block collimator, leading to much reduced leakage and significantly reduced complexity and weight in the radiation head. The block and the array of individually moveable elongate narrow leaves can be the only collimation apparatus in the head, offering weight reductions and height reductions relative to known radiation heads.

A radiotherapy control computer will usually be necessary, capable of controlling the radiation source and the collimation apparatus and programmable with an IMRT or IMRT-based treatment plan.

The block is preferably supported from the first side of the beam axis, and the array of individually moveable elongate narrow leaves is preferably supported from the second side of the beam axis.

The block can comprise an adjustable section defining its leading edge, capable of adjustment so as to vary the angle between the leading edge and the beam axis. This allows the leading edge of the block to be aligned to the local beam direction regardless of how far into the beam the block is positioned. This reduces the penumbra of the block and creates a better definition of the radiation field.

Alternatively, the block can comprise a plurality of plates stacked in the direction of the beam axis and moveable relative to each other so that the leading edge of the thus-defined block is adjustable so as to vary the angle between the leading edge and the beam axis.

This also avoids the difficulties that can arise through the use of interdigitation in a two-sided MLC, such as “tongue and groove” effects where a single leaf is sent into an aperture defined by the opposing bank, or the opposite situation of a single leaf opening where in- and out-spread of secondary electrons entails a significant local widening of the penumbra.

### BRIEF DESCRIPTION OF THE DRAWINGS

An embodiment of the present invention will now be described by way of example, with reference to the accompanying figures in which;

FIG. 1 shows the general layout of a known radiotherapy apparatus;

FIG. 2 shows a typical bank of leaves for an MLC;

FIG. 3 shows a beam's-eye view of an MLC and its associated block collimators;

FIG. 4 shows a first way of delivering a non-uniform dose pattern via an MLC;

FIG. 5 shows a second way of delivering a non-uniform dose pattern via an MLC;

FIG. 6 shows a view of part of the collimator set according to the present invention;

FIG. 7 shows the collimator set of FIG. 6, viewed along the beam direction;

FIG. 8 shows the collimator set of FIG. 6, viewed from one side;

FIG. 9 shows a second embodiment of the collimator set, viewed from one side; and

FIG. 10 shows a third embodiment of the collimator set, viewed from one side.

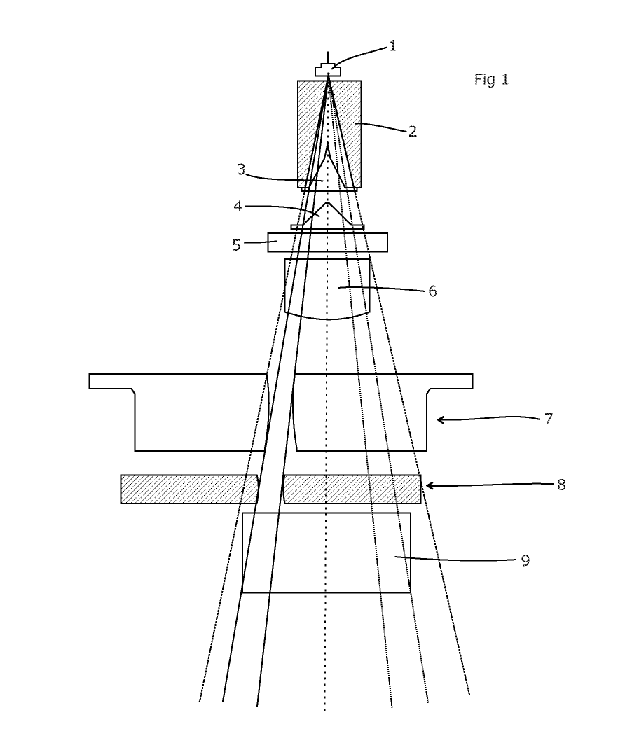

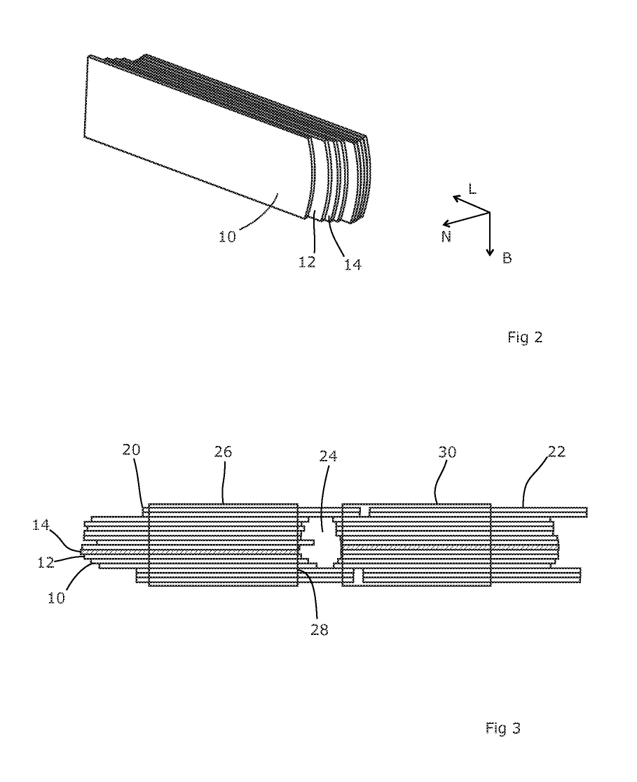

### DETAILED DESCRIPTION OF THE EMBODIMENTS

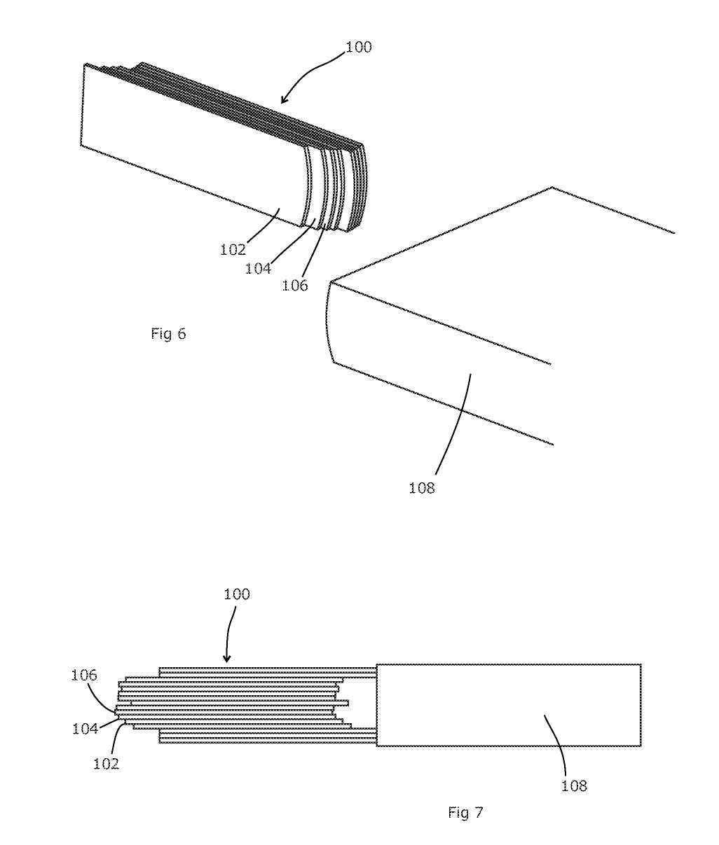

Referring to FIG. 6, the collimator set of the present invention for use in a radiotherapy apparatus comprising left and right collimators, each projecting into the beam from opposing sides of the apparatus. The left collimator 100 is a multi-leaf collimator as described above, and comprises a large number of leaves 102, 104, 106, some of which are illustrated and some of which are omitted for clarity. As discussed above, the left collimator may have 40, 80 or 160 leaves, or any other number.

The right collimator 108 is a single block collimator. Its cross-section in a plane parallel to the MLC leaves is substantially the same as the individual leaves, but it is in the form of a single block that extends across the entire width of the beam aperture.

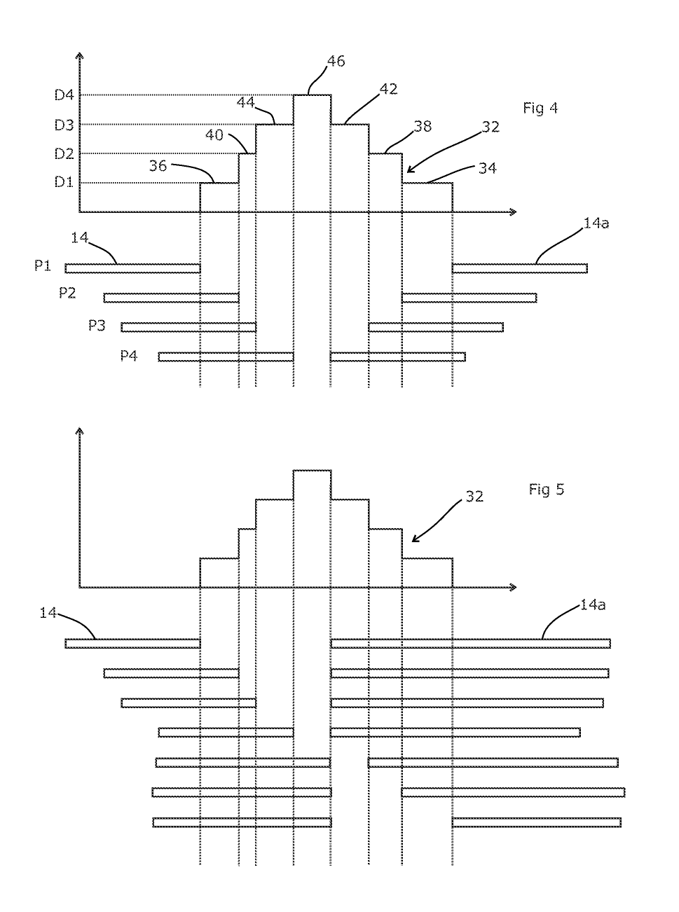

FIG. 7 shows the collimator set, viewed along the beam. The left collimator 100 is able to define an arbitrary shape subject only to the usual limitations of an MLC leaf bank. The right collimator 108 can define a straight edge, at any chosen point in the radiation field. Clearly, this is unsuited to most radiotherapy applications, i.e. those where the tumour does not have a straight edge. It may be suited to some applications, such as a tumour close to a linear structure such as the spinal cord. However, the collimator set is particularly applicable to IMRT-based applications, where the instantaneous radiation field does not necessarily conform to the shape of the tumour but is built up from a number of smaller fields (as described in relation to FIGS. 4 and 5 above). In such an application, the individual smaller fields need to be time-synchronised so that their right-hand edges are aligned, and can thus be provided by the block collimator.

By a suitable adjustment of the programs of the treatment planning computer and the radiotherapy control computer, this is straightforward to achieve. As a result, significant complexity in the radiation head is removed, and a significant amount of weight in the head can be eliminated. This will have obvious benefits in terms of the leakage radiation, the apparatus cost (both in acquisition and maintenance), and benefits in the design of other parts if the weight of the head that needs to be supported is reduced.

Other benefits can be obtained in terms of the quality of the penumbra that can be achieved. The 80/20 penumbra provided by a standard, single-focusing MLC is typically 7 mm (measured on a 6 MV device with a 10×10 cm field). For brain and spine radiosurgery, a more narrow penumbra, preferably 3 mm, is required, in order to create concave iso-dose surfaces with the very steep fall-off in dose that is required to protect nerves embedded in tumour tissue, for example the acoustic nerve or the spinal cord.

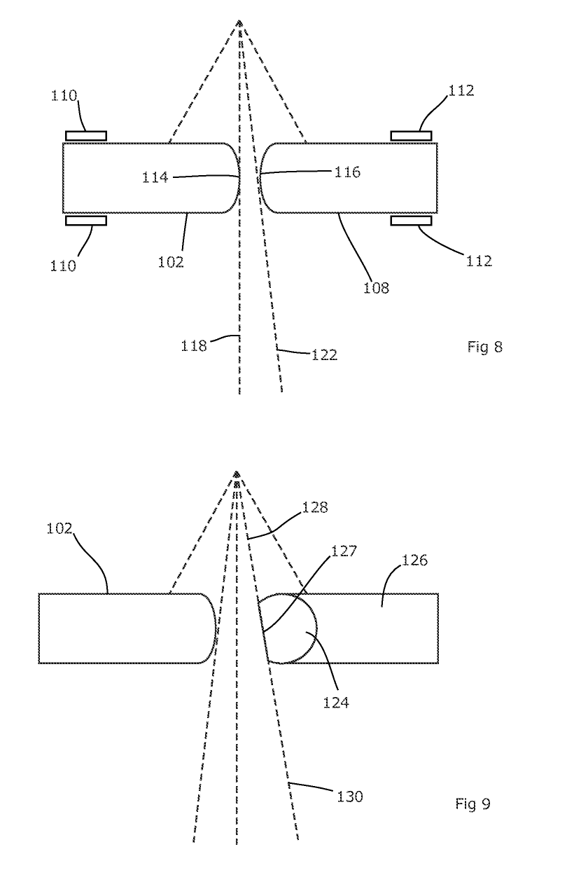

To provide a 3 mm penumbra the single-focusing design of a standard MLC is not appropriate, as leaf tips on such a unit must be rounded in order to achieve a consistent penumbra across field aperture. Typical leaf tips are shown in FIG. 8 on both the left-hand leaf 102 and the right-hand block 108. Both are supported in suitable frames 110, 112 respectively. Each has a rounded tip 114, 116 so that the penumbra caused by only partial attenuation of the beam at the tip of the leaf is uniform across the radiation field. This is a compromise; a straight edge would provide a very narrow penumbra, but only when the local beam direction at the tip was exactly aligned with the edge. At all other positions of the leaf or block, the edge would project into the beam and at the edge of the radiation field the beam would only be attenuated by the corner of the leaf or block. Thus, there would be an unacceptably wide penumbra. To avoid this, a curved profile as shown in FIG. 8 is usually provided, which has a relatively uniform penumbra across the whole field.

A so-called double-focusing collimator provides a much better penumbra, as the flat leaf tip can align with the beam trajectory no matter the leaf position. The downside is that a double-focusing unit is even more complicated and expensive to produce. By replacing one leaf bank with a block collimator (as in the present invention), we can align the edge with the beam trajectory and create a much narrower penumbra on one side of the treatment field, while still being able to apply full intensity modulation.

Thus, the single-focusing design of FIG. 8 is simplest in that it can utilise existing leaf bank designs. In its most basic design the block has a rounded surface similar to the MLC leaf tip. This design will reduce cost but not improve the penumbra. A normal penumbra is provided to both edges 118, 122 of the radiation field.

FIG. 9 shows a second embodiment in which a rotatable “log” 124 is fitted in the revised block collimator 126. The log 124 consists of a rotatable axle with a flat surface 127 which can be aligned with the beam trajectory 128, to provide a much narrower penumbra, across entire collimator aperture. The local beam direction 128 will be a one-to-one function of the position of the block 126 within the radiation field and can therefore be controlled mechanically or via a servo under control of the radiotherapy control computer or a local control device. As a result, the penumbra 130 on that side of the treatment field can be dramatically improved across the entire collimator aperture.

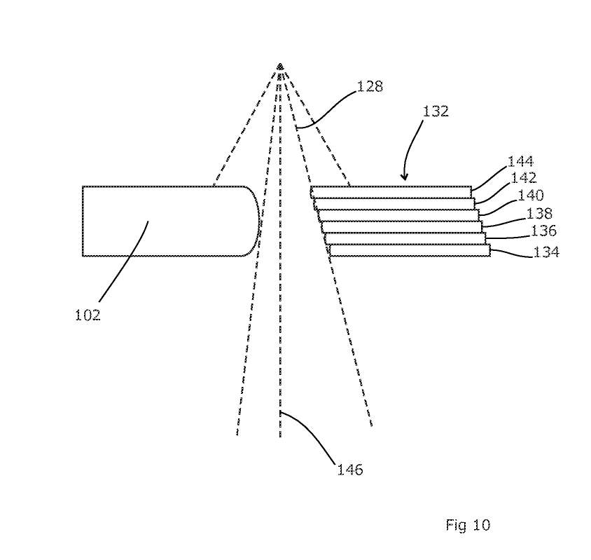

A third design shown in FIG. 10 uses a block 132 a stack of six movable plates 134, 136, 138, 140, 142, 144 which can be moved to align with the local beam trajectory 128 to provide a narrow penumbra across the collimator aperture. As illustrated, the plates are stacked vertically relative to the beam direction 146 and moveable over each other under mechanical or servo control as above. As each plate can be moved individually, the edge of the stack can be aligned with the beam trajectory and hence create a more narrow penumbra.

As illustrated in FIG. 10, the tips of the plates 134-144 can be rounded in order to better align with the beam trajectory. Alternatively, the tips can be straight, for example vertical.

Generally, the block, plates and “log” etc., should be made out of a high-Z material such as Tungsten, to achieve the best possible beam shaping properties.

As a result of the present invention, the same or better beam resolution and modulation capability can be achieved with the half number of collimator leaves. Compared with a traditional MLC, the collimators described above will reduce cost dramatically during production, assembly, installation, tuning and maintenance. A much steeper penumbra can be created on one side of the treatment field, which is required for brain and spine radiosurgery. The total leakage radiation dose caused by the typical leakage between the collimator leaves in a traditional MLC can be reduced by approximately 50%. With the “stack” concept of FIG. 10, a more narrow penumbra can be achieved on one side of the treatment field, which can be used to create steeper gradients required for certain radiosurgery applications, for example spine radiosurgery.

It will of course be understood that many variations may be made to the above-described embodiment without departing from the scope of the present invention.

1. Radiotherapy apparatus comprising;
a radiation source capable of emitting a beam of therapeutic radiation along a beam axis;
collimation apparatus for delimiting the beam, comprising;
i. a block of sufficient width to extend across the width of the beam, and selectively movable into the beam from a first side of the beam axis;
ii. an array of individually moveable elongate narrow leaves arranged side-by-side in a direction perpendicular to the beam, and each being moveable longitudinally into the beam from a second and opposing side of the beam axis;
wherein the collimation apparatus has no array of individually moveable elongate narrow leaves arranged side-by-side in a direction perpendicular to the beam moveable longitudinally into the beam from the first side of the beam axis.

2. Radiotherapy apparatus according to claim 1, wherein the block and the array of individually moveable elongate narrow leaves are the only collimation apparatus.

3. Radiotherapy apparatus according to claim 1, further comprising a radiotherapy control computer capable of controlling the radiation source and the collimation apparatus, and programmable with a treatment plan based on Intensity Modulated Radio Therapy (“IMRT”).

4. Radiotherapy apparatus according to claim 1, wherein the block is supported from the first side of the beam axis.

5. Radiotherapy apparatus according to claim 1, wherein the array of individually moveable elongate narrow leaves is supported from the second side of the beam axis.

6. Radiotherapy apparatus according to claim 1, wherein the block comprises an adjustable section which defines its leading edge, and wherein the block is capable of adjustment so as to vary the angle between the leading edge and the beam axis.

7. Radiotherapy apparatus according to claim 1, wherein the block comprises a plurality of plates stacked in the direction of the beam axis and moveable relative to each other so that the leading edge of the thus-defined block is adjustable so as to vary the angle between the leading edge and the beam axis.
